(19) United States
(12) Patent Application Publication
(10) Pub. No.: US 2022/0022394 A1
(43) Pub. Date: Jan. 27, 2022

(54) Aseptic Sowing And Raising Seedling Method For Distant Hybridization Seeds Of Phalaenopsis And Rhynchostylis Retusa

(71) Applicant: Environmental Horticulture Research Institute of Guangdong Academy of Agricultural Sciences, Guangzhou City (CN)

(72) Inventors: Heming Chen, Guangzhou City (CN); Fubing Lv, Guangzhou City (CN); Wenfang Xiao, Guangzhou City (CN); Zuo Li, Guangzhou City (CN)

(21) Appl. No.: 17/183,521

(22) Filed: Feb. 24, 2021

(30) Foreign Application Priority Data
Jul. 24, 2020 (CN) 202010720284.7

(51) Int. Cl. A01H 1/02; A01D 46/00; A01C 7/00; A01G 9/24; A01G 9/029; A01H 4/00

(52) U.S. Cl. CPC A01H 1/021; A01D 46/00; A01H 4/008; A01G 9/246; A01G 9/0293; A01C 7/00

(57) ABSTRACT

The disclosure provides an aseptic sowing and raising seedling method for distant hybridization seeds of Phalaenopsis and Rhynchostylis retusa, which takes Phalaenopsis as female parent and Rhynchostylis retusa as male parent to obtain hybridization fruit pods by artificial pollination, the fruit pods are pretreated, and the seeds are aseptically sowed, germinated, protocorm proliferated and differentiated, strong seedlings rooted, acclimatized and transplanted to obtain distant hybridization offspring groups. The disclosure has the advantages of high seed germination rate, large seedling number, short seedling time and good seedling quality, which solves the problems that the seeds of Phalaenopsis distant hybridization process are difficult to succeed due to affinity, and are extremely difficult to germinate and raise seedlings under natural conditions. Therefore, a large number of seedlings can be obtained in a short period by aseptic sowing and artificial propagation, which lays a foundation for breeding excellent varieties of Phalaenopsis distant hybridization.

### CROSS REFERENCE TO RELATED APPLICATION

This application claims the priority of Chinese Patent Application NO. 202010720284.7, entitled “aseptic sowing and raising seedling method for distant hybridization seeds of Phalaenopsis and Rhynchostylis retusa” filed with China National Intellectual Property Administration on Jul. 24, 2020, which is incorporated herein by reference in its entirety.

### TECHNICAL FIELD

The disclosure belongs to the technical field of tissue culture and rapid propagation, and particularly relates to an aseptic sowing and raising seedling method for distant hybridization seeds of Phalaenopsis and Rhynchostylis retusa.

### BACKGROUND ART

Phalaenopsis has the reputation of “Queen of Orchids” because of its peculiar flower shape, gorgeous colors, color richness, neat inflorescences and long flowering period. Phalaenopsis is currently the most sold orchid species in the world, Phalaenopsis accounts for 75% of all orchid sales in the United States, and ranks first among potted flowers in Netherlands, potted orchids in Japan and lunar new year flowers in China, with the annual global consumption exceeding 280 million. In recent years, the hybrid breeding of Phalaenopsis has made great achievements, which makes great contributions to industrial development, but the genetic basis of Phalaenopsis is still relatively narrow. Therefore, it is of great theoretical and practical significance to introduce excellent genes of intergeneric wild germplasm resources into Phalaenopsis, enrich the gene pool of Phalaenopsis and improve the resistance of Phalaenopsis.

Rhynchostylis retusa, a perennial plant of Orchidaceae, is a related genus of Phalaenopsis. Rhynchostylis retusa is originated in Guizhou and Yunnan, and is widely distributed in tropical Asia, from Sri Lanka and India to tropical Himalayas, through Laos, Vietnam, Cambodia, Malaysia to Indonesia and the Philippines. The plant has developed and thick aerial roots, with a thickness of 6-16 mm; stems are erect or oblique, usually 3-10 cm long, sometimes longer, unbranched, with few to many nodes, densely covered with overlapped leaf sheaths; the leaves are fleshy, in two rows, close to each other, curved outward and broad-banded; inflorescences are axillary, usually with 1-3 inflorescences, longer than or nearly as long as leaves, unbranched, usually drooping; flowers are white and densely covered with purple spots, spreading and papery. Rhynchostylis retusa is born in the sparse forest at an altitude of 310-1400 meters or on the trunk of the border tree, the flowering period is from May to June, and the plant has strong disease resistance, and high ornamental value and utilization value.

There exists compatibility barrier in distant hybridization of Phalaenopsis, wherein intergeneric hybrid embryos are usually stunted, and the seeds have no endosperm, so it is extremely difficult to germinate and raise seedlings under natural conditions. Therefore, a large number of seedlings can be germinated and obtained in a short time by aseptic sowing. At present, there is no research report on aseptic sowing and raising seedling method for distant hybridization seeds of Phalaenopsis and Rhynchostylis retusa. Therefore, the germination and seedling characteristics of distant hybridization seeds between Phalaenopsis and Rhynchostylis retusa are studied in order to lay a foundation for distant hybridization breeding and new variety cultivation between Phalaenopsis and Rhynchostylis retusa.

### SUMMARY OF THE INVENTION

In view of this, the disclosure provides an aseptic sowing and raising seedling method for distant hybridization seeds of Phalaenopsis and Rhynchostylis retusa. By adopting distant hybridization of Phalaenopsis and Rhynchostylis retusa, a large number of seedlings can be obtained in a short period, which lays a foundation for breeding excellent varieties of Phalaenopsis and Rhynchostylis retusa, and cultivates new orchid varieties, and provides an effective way for the production of distant hybridization seedlings of Phalaenopsis and Rhynchostylis retusa.

According to the disclosure, the technical problems above are solved by the following technical scheme:

An aseptic sowing and raising seedling method for distant hybridization seeds of Phalaenopsis and Rhynchostylis retusa, comprising:

(1) artificial pollination: taking Phalaenopsis as the female parent and Rhynchostylis retusa as the male parent for hybridization, selecting fresh and vigorous flowers, removing pollen mass on the lip and gynostemium of the female parent with sterilized tweezers, and taking fresh pollen mass of the male parent and placing them in the pistil cavity of the female parent; after pollination for 100-120 days, harvesting the uncracked mature fruit pods for aseptic sowing;

(2) pretreatment of fruit pods and seed sowing: washing the fruit pods with flowing water, scrubbing the fruit pods with 75% alcohol on an ultra-clean bench, sterilizing the fruit pods with 0.1% HgCl₂ solution for 10-12 min, washing the fruit pods with sterile water for 3-5 times, cutting the fruit pods after absorbing residual water with sterile filter paper, and sowing the seeds on germination medium for cultivation;

(3) seed germination: sowing the seeds on the germination medium for cultivation at 25-28° C., with dark or scattered light in the early stage, light intensity of 1000-1500 lx in the later stage and illumination time of 12 h/d;

(4) protocorm proliferation and differentiation: after the seeds germinating into peak green protocorm, transferring the protocorm to proliferation and differentiation culture medium for cultivation at 25-28° C., with light intensity of 1500-2000 lx and illumination time of 12 h/d;

(5) rooting culture of strong seedlings: carefully cutting off the seedlings differentiated from protocorm, and transferring the seedlings to rooting medium for strong seedlings at 25-28° C., with light intensity of 1500-2000 lx and illumination time of 12 h/d;

(6) acclimatization and transplantation: when there are 2-4 leaves, with leaf length of 3-5 cm, leaf width of 1-2 cm, and 2-4 roots, with root length of 2-5 cm, putting the seedlings into a greenhouse with natural light scattering for acclimatization, the temperature of the acclimatization greenhouse is controlled at 22° C.-28° C., and the seedling can be transplanted out of the bottle after acclimating for 20-30 days; keeping the planting temperature at 22-28 ° C. and humidity at 60-70%.

In some embodiments, the germination medium is Hyponex: 3.0 g/L+6-BA(6-benzylaminoadenine) 2.0 mg/L+NAA (naphthalene acetic acid) 1.0 mg/L+10.0% coconut milk+sucrose 30 g/L+agar 7 g/L, with the pH of 5.5-5.8.

In some embodiments, the proliferation and differentiation medium is Hyponex: 3.0 g/L+6-BA(6-benzylaminoadenine) 2.0 mg/L+Ad(adenine) 2.0 mg/L+10.0% coconut milk+sucrose 30 g/L+agar 7 g/L, with the pH of 5.5-5.8.

In some embodiments, the rooting medium for strong seedlings is Hyponex: 3.0 g/L+NAA (naphthalene acetic acid) 0.5 mg/L+10.0% coconut milk+sucrose 30 g/L+agar 7 g/L, with the pH of 5.5-5.8.

In some embodiments, the transplantation in step (6) specifically comprises: taking off the bottle stopper when discharging the bottle, clamping the tissue culture seedlings out of the bottle with tweezers, cleaning the attached culture medium in clear water, sterilizing in 0.1% potassium permanganate solution for 2-3 min, taking out and drying with absorbent paper such as newspaper, and directly planting in a transparent plastic cup with water moss.

Compared with the prior art, the aseptic sowing and raising seedling method for distant hybridization seeds of Phalaenopsis and Rhynchostylis retusa has the following beneficial effects:

(1) the proliferation and differentiation of protocorm are realized by using the same formula medium (Hyponex: 3.0 g/L+6-BA 2.0 mg/L+Ad 2.0 mg/L+10.0% coconut milk+sucrose 30 g/L+agar 7 g/L), which reduces the bottle-transferring process, shortens the seedling time, reduces the pollution rate and improves the seedling rate.

(2) appropriate amount of coconut milk is added throughout the cultivation process, which is conducive to seed germination, promotes protocorm proliferation and strong seedling rooting.

(3) acclimating the seedlings in the greenhouse for 20-30 days before transplanting, which is conducive for bottle seedlings to adapt to the environment of greenhouse, thus improving the survival rate of seedling transplanting.

In summary, the disclosure has the advantages of high seed germination rate, large seedling number, short seedling time and good seedling quality, and the survival rate of seedling transplantation reaches 95.0%, which solves the problems that the seeds of Phalaenopsis distant hybridization process are difficult to succeed due to affinity, and are extremely difficult to germinate and raise seedlings under natural conditions, which lays a foundation for breeding excellent varieties of Phalaenopsis distant hybridization.

The disclosure takes Phalaenopsis Jiuhbao Red Rose as the female parent and Rhynchostylis retusa as the male parent to carry out intergeneric distant hybridization breeding, thereby solving the key technical problems of propagation and seedling raising of distant hybridization seeds such as aseptic sowing, seed germination, protocorm proliferation and differentiation, strong seedling rooting, acclimatization and transplantation, and obtaining a large number of hybrid offspring plants, which lays a foundation for the breeding of distant hybridization excellent varieties.

### DETAILED DESCRIPTION OF THE EMBODIMENTS

In view of this, the disclosure provides an aseptic sowing and raising seedling method for distant hybridization seeds of Phalaenopsis and Rhynchostylis retusa, comprising:

(1) artificial pollination: taking Phalaenopsis as the female parent and Rhynchostylis retusa as the male parent for hybridization, selecting fresh and vigorous flowers, removing pollen mass on the lip and gynostemium of the female parent with sterilized tweezers, and taking fresh pollen mass of the male parent and placing them in the pistil cavity of the female parent; after pollination for 100-120 days, harvesting the uncracked mature fruit pods for aseptic sowing;

(2) pretreatment of fruit pods and seed sowing: washing the fruit pods with flowing water, scrubbing the fruit pods with 75% alcohol on an ultra-clean bench, sterilizing the fruit pods with 0.1% HgCl₂ solution for 10-12 min, washing the fruit pods with sterile water for 3-5 times, cutting the fruit pods after absorbing residual water with sterile filter paper, and sowing the seeds on germination medium for cultivation;

(3) seed germination: sowing the seeds on the germination medium for cultivation at 25-28° C., with dark or scattered light in the early stage, light intensity of 1000-1500 lx in the later stage and illumination time of 12 h/d;

(4) protocorm proliferation and differentiation: after the seeds germinating into peak green protocorm, transferring the protocorm to proliferation and differentiation culture medium for cultivation at 25-28° C., with light intensity of 1500-2000 lx and illumination time of 12 h/d;

(5) rooting culture of strong seedlings: carefully cutting off the seedlings differentiated from protocorm, and transferring the seedlings to rooting medium for strong seedlings at 25-28° C., with light intensity of 1500-2000 lx and illumination time of 12 h/d;

(6) acclimatization and transplantation: when there are 2-4 leaves, with leaf length of 3-5 cm, leaf width of 1-2 cm, and 2-4 roots, with root length of 2-5 cm, putting the seedlings into a greenhouse with natural light scattering for acclimatization, the temperature of the acclimatization greenhouse is controlled at 22° C.-28° C., and the seedling can be transplanted out of the bottle after acclimating for 20-30 days; keeping the planting temperature at 22-28° C. and humidity at 60-70%.

The treatment methods, cultivation conditions, cultivation time and culture medium components involved in the above steps will be appropriately adjusted according to specific needs.

In some embodiments, the germination medium is Hyponex: 3.0g/L+6-BA(6-benzylaminoadenine) 2.0 mg/L+NAA (naphthalene acetic acid) 1.0 mg/L+10.0% coconut milk+sucrose 30 g/L+agar 7 g/L, with the pH of 5.5-5.8.

In some embodiments, the proliferation and differentiation medium is Hyponex: 3.0 g/L+6-BA(6-benzylaminoadenine) 2.0 mg/L+Ad(adenine) 2.0 mg/L+10.0% coconut milk+sucrose 30 g/L+agar 7 g/L, with the pH of 5.5-5.8.

In some embodiments, the rooting medium for strong seedlings is Hyponex: 3.0 g/L+NAA (naphthalene acetic acid) 0.5 mg/L+10.0% coconut milk+sucrose 30 g/L+agar 7 g/L, with the pH of 5.5-5.8.

In some embodiments, the transplantation in step (6) specifically comprises: taking off the bottle stopper when discharging the bottle, clamping the tissue culture seedlings out of the bottle with tweezers, cleaning the attached culture medium in clear water, sterilizing in 0.1% potassium permanganate solution for 2-3 min, taking out and drying with absorbent paper such as newspaper, and directly planting in a transparent plastic cup with water moss.

In addition, the cultivation process is influenced by various factors such as temperature, illumination, humidity, etc., therefore in each step of the disclosure, the processing mode, cultivation conditions and cultivation time can be appropriately adjusted according to specific needs.

In order to make the present disclosure easier to understand, specific embodiments of the present disclosure will be further explained below.

The Phalaenopsis Jiuhbao Red Rose and Rhynchostylis retusa was taken as the female parent and male parent respectively to carry out intergeneric distant hybridization breeding, aseptic sowing and seedling propagation of hybridization seeds, and seedling raising , comprising:

(1) the Phalaenopsis Jiuhbao Red Rose and Rhynchostylis retusa was taken as the female parent and male parent respectively to carry out distant hybridization, fresh and vigorous flowers were selected, pollen mass on the lip and gynostemium of the female parent was removed with sterilized tweezers, and the fresh pollen mass of the male parent was taken and placed in the pistil cavity of the female parent; after pollination for 100-120 days, the uncracked mature fruit pods were harvested for aseptic sowing;

(2) pretreatment of fruit pods and seed sowing: the fruit pods were washed with flowing water, scrubbed with 75% alcohol on an ultra-clean bench, sterilized with 0.1% HgCl₂ solution for 10-12 min, and washed with sterile water for 3-5 times, the residual water on the fruit pods was absorbed with sterile filter paper, the fruit pod was hold with tweezers in one hand, and cut longitudinally with the sterilized scalpel in the other hand to expose the seeds, and then another tweezers was used to directly grab the seeds and spread them on the germination medium; wherein the germination medium is Hyponex: 3.0 g/L+6-BA(6-benzylaminoadenine) 2.0 mg/L+NAA (naphthalene acetic acid) 1.0 mg/L+10.0% coconut milk+sucrose 30 g/L+agar 7 g/L, with the pH of 5.5-5.8.

(3) seed sowing: the seeds were sowed on the germination medium for cultivation at 25-28° C., with dark or scattered light in the early stage, light intensity of 1000-1500 lx in the later stage and illumination time of 12 h/d; the seeds started to expand after 30 days of culture, and after about 60 days, they expanded to form white bulbous stems, after 30 days of continuous culture, the embryos were germinated into peak green protocorms;

(4) protocorm proliferation and differentiation: the protocorm germinated to 3-5 mm was transferred to the proliferation and differentiation medium for culture, which was Hyponex: 3.0 g/L+6-BA(6-benzylaminoadenine) 2.0 mg/L+Ad(adenine) 2.0 mg/L+10.0% coconut milk+sucrose 30 g/L+agar 7 g/L, with the culture temperature of 25-28° C., with light intensity of 1500-2000 lx and illumination time of 12 h/d; after culture for 100d, the multiplication coefficient reached 3.1;

(5) rooting culture of strong seedlings: the seedlings, with a height of 2-4 cm, differentiated from protocorm were carefully cut off and transferred to the rooting medium for strong seedlings, which is Hyponex: 3.0 g/L+NAA (naphthalene acetic acid) 0.5 mg/L+10.0% coconut milk+sucrose 30 g/L+agar 7 g/L, with the culture temperature of 25-28° C., with light intensity of 1500-2000 lx and illumination time of 12 h/d; after culture for 100d, the seedling rooting rate reached 93.7%;

(6) acclimatization and transplantation: when there were 2-4 leaves, with leaf length of 3-5 cm, leaf width of 1-2 cm, and 2-4 roots, with root length of 2-5 cm, the seedlings were put into a greenhouse with natural light scattering for acclimatization, the temperature of the acclimatization greenhouse was controlled at 22° C.-28° C., and the seedling could be transplanted out of the bottle after acclimating for 20-30 days; the bottle stopper was taken off when discharging the bottle, the tissue culture seedlings were clamped out of the bottle with tweezers and cleaned the attached culture medium in clear water, then sterilized in 0.1% potassium permanganate solution for 2-3 min, taken out and dried with absorbent paper such as newspaper, and directly planted in a transparent plastic cup with water moss; the planting temperature was kept at 22-28° C. and humidity was kept at 60-70%; after planting for 2 days, the blade face was sprayed with water and fungicide(such as 0.1% carbendazim), and then conventional cultivation and management were carried out, the survival rate of seedlings reached 95.0% after transplanting for 60 days.

Compared with the prior art, the aseptic sowing and raising seedling method for distant hybridization seeds of Phalaenopsis and Rhynchostylis retusa has the following beneficial effects:

(1) the proliferation and differentiation of protocorm are realized by using the same formula medium (Hyponex: 3.0 g/L+6-BA 2.0 mg/L+Ad 2.0 mg/L+10.0% coconut milk+sucrose 30 g/L+agar 7 g/L), which reduces the bottle-transferring process, shortens the seedling time, reduces the pollution rate and improves the seedling rate.

(2) appropriate amount of coconut milk is added throughout the cultivation process, which is conducive to seed germination, promotes protocorm proliferation and strong seedling rooting.

(3) acclimating the seedlings in the greenhouse for 20-30 days before transplanting, which is conducive for bottle seedlings to adapt to the environment of greenhouse, thus improving the survival rate of seedling transplanting.

In summary, the disclosure has the advantages of high seed germination rate, large seedling number, short seedling time and good seedling quality, and the survival rate of seedling transplantation reaches 95.0%, which solves the problems that the seeds of Phalaenopsis distant hybridization process are difficult to succeed due to affinity, and are extremely difficult to germinate and raise seedlings under natural conditions, which lays a foundation for breeding excellent varieties of Phalaenopsis distant hybridization.

The disclosure takes Phalaenopsis Jiuhbao Red Rose as the female parent and Rhynchostylis retusa as the male parent to carry out intergeneric distant hybridization breeding, thereby solving the key technical problems of propagation and seedling raising of distant hybridization seeds such as aseptic sowing, seed germination, protocorm proliferation and differentiation, strong seedling rooting, acclimatization and transplantation, and obtaining a large number of hybrid offspring plants, which lays a foundation for the breeding of distant hybridization excellent varieties.

The above described are only preferred embodiments of the present application, It should be understood by those skilled in the art that, without departing from the principle of the present application, any variations and modifications fall into the scope of the present application.

1. An aseptic sowing and raising seedling method for distant hybridization seeds of Phalaenopsis and Rhynchostylis retusa, comprising:
(a) artificial pollination: taking Phalaenopsis as female parent and Rhynchostylis retusa as male parent for hybridization, selecting fresh and vigorous flowers, removing pollen mass on lip and gynostemium of the female parent with sterilized tweezers, and taking fresh pollen mass of the male parent and placing them in pistil cavity of the female parent; after pollination for 100-120 days, harvesting uncracked mature fruit pods for aseptic sowing;
(b) pretreatment of fruit pods and seed sowing: washing the fruit pods with flowing water, scrubbing the fruit pods with 75% alcohol on an ultra-clean bench, sterilizing the fruit pods with 0.1% HgCl₂ solution for 10-12 min, washing the fruit pods with sterile water for 3-5 times, cutting the fruit pods after absorbing residual water with sterile filter paper, and sowing the seeds on germination medium for cultivation;
(c) seed germination: sowing the seeds on the germination medium for cultivation at 25-28° C., with dark or scattered light in early stage, light intensity of 1000-1500 lx (cool-white fluorescent) in later stage and illumination time of 12 h/d;
(d) protocorm proliferation and differentiation: after the seeds germinating into peak green protocorm, transferring the protocorm to proliferation and differentiation culture medium for cultivation at 25-28° C., with light intensity of 1500-2000 lx (cool-white fluorescent) and illumination time of 12 h/d;
(e) rooting culture of strong seedlings: carefully cutting off the seedlings differentiated from protocorm, and transferring the seedlings to rooting medium for strong seedlings at 25-28° C., with light intensity of 1500-2000 lx (cool-white fluorescent) and illumination time of 12 h/d; and
(f) acclimatization and transplantation: when there are 2-4 leaves, with leaf length of 3-5 cm, leaf width of 1-2 cm, and 2-4 roots, with root length of 2-5 cm, putting the seedlings into a greenhouse with natural light scattering for acclimatization, temperature of acclimatization greenhouse is controlled at 22° C.-28° C., and the seedling can be transplanted out of a bottle after acclimating for 20-30 days; keeping the planting temperature at 22-28° C. and humidity at 60-70%;
wherein the germination medium is Hyponex®: 3.0 g/L+6-BA(6-benzylaminoadenine) 2.0 mg/L+NAA (naphthalene acetic acid) 1.0 mg/L+10.0% coconut milk+sucrose 30 g/L+agar 7 g/L, with the pH of 5.5-5.8;
wherein the proliferation and differentiation medium is Hyponex®: 3.0 g/L+6-BA(6-benzylaminoadenine) 2.0 mg/L+Ad(adenine) 2.0 mg/L+10.0% coconut milk+sucrose 30 g/L+agar 7 g/L, with the pH of 5.5-5.8; and
wherein the rooting medium for strong seedlings is Hyponex®: 3.0 g/L+NAA (naphthalene acetic acid) 0.5 mg/L+10.0% coconut milk+sucrose 30 g/L+agar 7 g/L, with the pH of 5.5-5.8.

2-4. (canceled)

5. The method according to claim 1, wherein the transplantation in step (6) comprising: taking off a stopper of the bottle when discharging the bottle, clamping the tissue culture seedlings out of the bottle with tweezers, cleaning the attached culture medium in clear water, sterilizing in 0.1% potassium permanganate solution for 2-3 min, taking out and drying with absorbent paper, and directly planting in a transparent plastic cup with water moss.
